(19) United States
(12) Patent Application Publication
(10) Pub. No.: US 2021/0267929 A1
(43) Pub. Date: Sep. 2, 2021

(54) COMPOSITIONS AND METHODS FOR TREATMENT OF ORAL ULCERATION AND ORAL MUCOSITIS

(71) Applicant: CelaCare Technologies, Inc., Dallas, TX (US)

(72) Inventors: Kenneth M. Yates, Keller, TX (US); Celia A. Proctor, Lead Hill, AR (US); Daniel H. Atchley, Searcy, AR (US)

(73) Assignee: CelaCare Technologies, Inc., Dallas, TX (US)

(21) Appl. No.: 17/257,386

(22) PCT Filed: Jul. 2, 2019
(86) PCT No.: PCT/US19/40226

§ 371 Date: Dec. 31, 2020

Related U.S. Application Data
(60) Provisional application No. 62/693,028, filed on Jul. 2, 2018.

(51) Int. Cl. A61K 31/28; A61K 47/32; A61K 47/36; A61K 47/18; A61P 1/04; A61K 9/00; A61K 9/08

(52) U.S. Cl. CPC A61K 31/28; A61K 47/32; A61K 47/36; A61K 9/08; A61P 1/04; A61K 9/0053; A61K 47/18

(57) ABSTRACT

An oral rinse including ionic silver as silver citrate complex and acemannan is useful for the treatment of oral ulceration and oral mucositis. The oral rinse has shown effectiveness against fungal and bacteria species such as Candida species, Staphylococcus aureus and Streptococcus pyogenes.

This application claims priority to U.S. Provisional Patent Application No. 62/693,028, entitled “Compositions and Methods for Treatment of Oral Ulceration and Oral Mucositis,” filed Jul. 2, 2018, the entire contents of which are hereby incorporated by reference.

### BACKGROUND

The present disclosure relates to compositions and methods for the treatment of oral ulceration and oral mucositis, including those that are associated with fungal and bacterial infections such as Candida, Staphylococcus and Streptococcus infections.

Silver has been utilized in multiple topical products for its broad spectrum antimicrobial activity against numerous microorganisms including multi-drug resistant microorganisms such as MRSA. However, high concentrations of silver are known to be cytotoxic which limits the use of silver products to less than 2 weeks.

A large molecular weight complex carbohydrate obtained from the inner leaf gel of Aloe vera, has been identified and given the United States Adopted Name, acemannan. Fundamentally, acemannan, an immunotherapeutic, can be separated from the inner leaf gel either by alcohol precipitation, column purification or ultra-filtration. Acemannan process for preparation and its uses have been described in U.S. Pat. Nos. 4,735,935, 4,851,224, 4,957,907, 4,959,214, 4,917,890, 4,966,892, 5,106,616, 5,118,673, 5,308,838, 5,441,943, 5,703,060, 5,760,102 and 5,902,796. Compositions and uses in combination with silver citrate salts have been described in U.S. Pat. Nos. 9,327,029 and 10,272,108, and European Patent Number 2704729. The entire contents of each patent are hereby incorporated by reference.

Multiple properties have been attributed to acemannan but the most predominant has been its immunomodulation function. Included as part of the immunomodulation property is the ability to stimulate release of primary growth factors necessary for optimal and accelerated wound healing. There is also some evidence that acemannan may interfere with adherence of bacteria to epithelia cells. In addition to its immune modulation activities it has also been shown to have anti-inflammatory properties and aid in the control of pain. The use of acemannan as a component of the present invention provides multiple attributes for its positive impact on healing, reduction of inflammation and pain control.

Acemannan has been utilized in marketed oral care medical devices with topical claims for management of aphthous ulcers, alveolar osteitis and oral stomatitis.

Mucositis is defined as inflammation of the mucosal surfaces throughout the body. Oral mucositis manifests as erythema, inflammation, ulceration, and hemorrhage in the mouth and throat. It often occurs as a complication of anticancer treatment and occurs in approximately 20-40% of patients receiving conventional chemotherapy, 80% of patients receiving high dose chemotherapy for HSCT, and in nearly 100% of patients receiving head and neck radiation therapy. Severe mucositis can require a dose reduction or treatment break from chemo or radiation therapy that can have a negative effect on patient cancer response and recovery. There are five stages of oral mucositis based on the World Health Organization's toxicity rating:
0: No symptoms
I (mild): Oral soreness and erythema
II (moderate): Oral erythema, ulcers, but solid diet tolerated
III (severe): Oral ulcers, liquid diet only
IV (life-threatening): oral alimentation impossible

There is a diverse pathogenesis of oral mucositis. Chemo- and Radiation therapy causes direct cell damage, which initiates a complex cascade of events that leads to oral mucositis. Reactive Oxygen Species, second messengers, pro-inflammatory cytokines and pathways, and metabolic by products are all believed to play a role in intensifying tissue damage in oral mucositis. Patients are also at an increased risk of developing bacterial and fungal infections due to initial tissue damage and over colonization of natural flora related to immunosuppression. Pseudomembranous candidiasis is commonly diagnosed in patients who are immunosuppressed. Candida albicans is one of the main opportunistic infections seen in these patients and up to 60% of immunocompetent individuals may harbor this organism in their oropharynx. It usually presents as white or yellow superficial plaques which represent an accumulation of yeast overgrowth, epithelial cell desquamation, keratin, bacteria, and necrotic tissue. The patient may be asymptomatic or could have a combination of any of the following symptoms: burning, itching, stinging and/or altered taste perception. C. albicans has also been shown to predispose the patient to other secondary infections. An animal model study, Kong et al, showed that mice with oral candidiasis (OC), after subsequent exposure to S. aureus, developed systemic bacterial infection with high morbidity and mortality. Those mice only exposed to either S. aureus or C. albicans did not show any signs of systemic disease although organisms were recovered from their tongues. Therefore, oral mucositis and candidiasis prevention is extremely important in immunocompromised patients and treatment should involve reducing or binding free radicals, modulating immune response as well as preventing secondary infections through decreased bacterial and fungal load.

Timely and efficacious treatment and management is extremely important since disease progression in severity can ultimately result in discontinuation of cancer therapies resulting in poor prognosis, as well as potentially leading to secondary infection and sepsis if left untreated. Panghal et al reported a study undertaken to determine incidence and risk factors for infection in oral cancer patients undergoing chemotherapy, radiation therapy, or both combined. They concluded that the most significant risk factor in the development of oral cavity infection in these patients was oral mucositis grade 3 and 4. They also observed that colonization of Candida albicans was the most significant oral cavity pathogen at 93% colonization with infection up to 30%, in those patients who received radiotherapy. The study also investigated septic infections that occurred in these patients and reported the most significant risk factor was nosocomial acquired infections, followed closely by mucositis grade 4 and central venous line infection. The disease ultimately can be very painful for patients that eventually can cause eating and drinking difficulties or in severe cases render it impossible.

At this time, there is only one drug approved by the FDA for treatment of severe oral mucositis, palifermin (Kepivance®, Biovitrum, Stockholm, Sweden). According to treatment guidelines it is recommended to use palifermin (human keratinocyte growth factor-1) to prevent oral mucositis in high risk patients receiving high dose chemotherapy with total body irradiation. Other treatments that are recommended by guidelines for prevention depending on anticancer treatment are cryotherapy, low level laser therapy, good oral hygiene or rinsing with sterile water, and benzydamine mouthwash. Additionally, other oral cavity moisturizing products are available to help manage dryness of the oral cavity associated with chemo- and radiation induced mucositis. Once oral mucositis has developed the treatments are mainly aimed at patient's symptoms and prevention of infection with physical barriers, opioids for pain, anti-inflammatories, and antibiotics. However, there is not one of these treatments that has been shown to be significantly better than the other, or one that is all encompassing, and the results vary from patient to patient because of the diverse pathogenesis. Treatment of oral mucositis is lacking and new developments that focus more on the pathology and risk of infection associated with oral mucositis could potentially improve patient outcomes.

### SUMMARY

The present disclosure relates to compositions and methods for the treatment of oral ulceration and oral mucositis including those that are associated with fungal and bacterial infections such as Candida, Staphylococcus and Streptococcus infections. The compositions are liquid compositions including a low concentration of ionic silver as a silver citrate complex salt and acemannan and are used as an oral rinse.

While high concentration of silver is known to be cytotoxic which limits the use of silver products to less than 2 weeks, the preferred silver concentration in the present compositions is about 10 ppm, which is at least 5 to 20 times lower than the silver concentration used in other over the counter topical medical device products. These range from 55 to 200 ppm in some examples. Also, the level of silver in the present compositions is too low to potentially cause argyria or permanent silver deposits in the skin or mucosal tissues. While the total amount of silver required to cause argyria is unknown, total body contents from past cases suggest a range of at least 3.8-6.4 g of silver. In addition, silver ranges in untreated drinking water range from barely detectable to 5 ppm. Drinking water treated with silver for disinfection purposes may have levels of silver up to 50 ppm. The present compositions containing about 10 ppm of silver fall well in the safe consumption range when compared to treated drinking water. Animal studies in mice, rats, dogs, and monkeys estimated human elimination of silver from oral ingestion at 90%. According to the EPA a safe lifetime continuous exposure of silver is 5 micrograms/kg/day, which would translate to 350 micrograms/day for a 70 kg patient. The silver content in the present compositions that a patient is exposed to per day is up to 400 micrograms. However, since the compositions are utilized as an oral rinse and are not swallowed, the potentially ingested silver is expected to be well below the safe lifetime daily exposure maximum.

Acemannan does not contain irritants that are commonly found as part of Aloe vera and known to cause skin irritation. The product has been evaluated for cytotoxicity and deemed to be non-cytotoxic using a standard protocol at a GLP laboratory. No significant toxicity was seen in mice, rats, or dogs at maximum dose levels of 200 and 50 mg/kg/day when administered IV or IP at 4-day intervals for 30 days. Also, doses of 1500 mg/kg/day in the diet of dogs and 2000 mg/kg/day in the diet of mice for 180 days had no observable adverse effects. A safety study for acemannan in humans was conducted to evaluate its effect in the treatment of oral aphthous ulcers. The initial study of 50 healthy patients received 0.5% acemannan incorporated into a Carbopol thickener applied to the oral mucosa of the lower lip three times a day for 7 days. Oral exams and kidney and liver function were evaluated prior to and at the end of the study. No patients reported adverse effects or allergic reactions with the administration of acemannan. Also, there was no statistical difference observed between pre- and post-test blood values. Since the present compositions are utilized as an oral rinse, acemannan is not expected to be absorbed systemically through the mucosa and will merely coat the patients' mouths. Acemannan has also previously been cleared to market in oral device products. Based on current safety data on the ingredients incorporated into the compositions and the 14-day duration of use for patients in the study, the compositions will not provide significant patient risks when dosed as intended.

In the current compositions, the combination of acemannan with silver citrate complex enhances silver's antimicrobial activity and makes it effective in the management of secondary infections from oral mucositis.

### DETAILED DESCRIPTION OF THE PREFERRED EMBODIMENTS

The current compositions and methods relate to the treatment of oral ulceration and oral mucositis.

In preferred embodiments, the present compositions include ionic silver at about 10 ppm as a silver citrate complex salt and acemannan at about 0.4 w/w %. Additional excipients include Disodium EDTA, Hyaluronic acid, Polyvinylpyrrolidone (PVP), purified water, a flavoring agent, and a buffer such as tris(hydroxymethyl)aminomethane to bring the pH of the composition to about 6 to 6.7. The pH of the oral mucosae found in literature is between 6.28-7.34 with the mean of 6.78. The compositions coat the oral mucosae exposing bacteria and yeast to ionic silver while also reducing inflammation, pain and discomfort allowing for mucosal healing.

In additional preferred embodiments, the compositions include silver citrate complex at about 5 to about 10 μg/mL, and acemannan at about 3 to about 5 mg/mL. In further preferred embodiments the compositions may include about 3 μg/mL silver citrate complex to about 15 μg/mL and about 1 mg/mL acemannan to about 10 mg/ml.

Silver citrate complex as used herein is a silver salt that is a stable mixture of citric acid monohydrate and silver dihydrogen citrate monohydrate providing for the stabilized silver citrate complex. Silver citrate complex is a broad-spectrum antimicrobial silver salt that has been demonstrated to have effect against gram-positive and gram-negative bacteria as well as against yeast and molds and its safety has been described in Health and Consumer Protection Directorate-General Report SCCP/1196/08. The use of silver citrate salts in compositions containing acemannan for treating wounds or lesions have been described in U.S. Pat. Nos. 9,327,029 and 10,272,108, and European Patent Number 2704729. The entire contents of the patent is hereby incorporated by reference. The use of silver citrate complex as a component in the present compositions is for its effect against Candida sp. as well as its broad spectrum anti-bacterial effects.

Disodium ethylenediaminetetraacetic acid (disodium EDTA) is included in the compositions as a stabilizer. A second stabilizer and dispersant that can be included in the composition is polyvinylpyrrolidone (PVP). Hyaluronic acid can be included as a mucosal coating agent. Further excipients that are present in preferred embodiments include flavoring agents, purified water, and a buffer such as tris(hydroxymethyl)aminomethane. These excipients are all known to be safe for oral administration. In one preferred embodiment, the composition includes 5 mg/mL Disodium EDTA, 0.1 mg/mL Hyaluronic acid, 2 mg/mL PVP, purified water, and sufficient tris(hydroxymethyl)aminomethane to adjust the pH to about 6 to 6.6.

In order to prepare the compositions there are several methods that would be acceptable to one knowledgeable and skilled in the art. One such method would consist of creating an acemannan phase followed by addition of the silver salt phase. Using a phased approach for composition preparation, the acemannan phase can be compounded at a level sufficient to provide the desired concentration of the final product.

In preferred embodiments, the compositions are administered orally by swishing in the oral cavity for about 30 seconds and then expectorating the material. This may be repeated on multiple occasions throughout the day, preferably about 4 times, including after meals and at bedtime. The treatment may be extended for a number of days and in certain preferred embodiments lasts for 14 days. In preferred embodiments, a dose of about 10 mL (or 2 tsp) is administered, which contains 100 μg of silver and 40 mg of acemannan.

### Example 1

In the examples below, ATCC means American Type Culture Collection, (Manassas, Va.), CAMHB means Cation-adjusted Mueller-Hinton Broth, and LHB means lysed horse blood.

The purpose of this experiment was to determine the potency of one embodiment of the oral rinse on different Candida species, namely Candida albicans_{ATCC} 10231 and ATCC 24433, Candida glabrata_{ATCC} 15126, Candida krusei (Issechenkia orientalis)_{ATCC} 6258, Candida parapsilosis_{ATCC} 22019, and Candida tropicalis_{ATCC} 1369, to determine the minimum inhibitory concentration (MIC) of the oral rinse on each species. The oral rinse was compared to current antifungal treatments on the market.

The embodiment of the oral rinse 150528 tested in this experiment included 10 μg/mL silver citrate complex, 3 mg/mL acemannan, 5 mg/mL Disodium EDTA, 0.1 mg/mL Hyaluronic acid, 2 mg/mL PVP, 3.5 mg/ml of masking and cherry flavor, purified water, pH adjustment with tris(hydroxymethyl)aminomethane to a pH of 6.59. The current antifungal treatments that were tested and compared were Fluconazole (Sigma-Aldrich, Cat: PHR1160-1G, Lot LRAA6502) a Nystatin suspension (Sigma-Aldrich, Cat: N6261-SMU, Lot 020M13491V), and Gentian Violet (1% Humco, Cat: NDC 0395-1003-92, Lot547845).

The antimicrobial activity of the oral rinse solution as described above, Example 1, was tested using the micro-tube dilution method described in the Method for Determination of Broth Dilution MICs of Antifungal agents for fermentative yeasts, published by the European Committee for Antimicrobial Susceptibility Testing (EUCAST). This method was used to determine the minimal inhibitory concentrations (MICs) for the antimicrobial agents listed in Table 1 below. In brief, a standard inoculum of each microorganism was prepared by diluting a 0.5 MacFarland turbidity standard of the microorganism 1:100 in RPMI media without sodium bicarbonate plus 2% glucose. Then, 100 μL of the standard inoculum was added to equal volumes of two-fold serial dilutions of the oral rinse solution or controls, and the plates were incubated at 35° C. for 48 hours. MICs were determined photometrically as the lowest antimicrobial agent concentration (last well in the dilution series) that inhibited OD530 absorbance at least 50%. Each experiment was performed three times, with each antimicrobial agent tested in duplicate or triplicate.

Table 1 below shows the average MIC for each of four tested products—oral rinse, Fluconazole, Nystatin, and Gentian Violet, against each of five different Candida species.

TABLE 1

| | | | Standard | |
|---|---|---|---|---|
| MIC (mg/L) | | Mean | Deviation | |
| Candida albicans | Fluconazole | 1.2 | 0.4 | N = 6 |
| ATCC 10231 | Nystatin | 3.9 | 1.7 | N = 9 |
| | Gentian Violet | 0.3 | 0.0 | N = 6 |
| | Oral rinse 150528 | 0.0007 | 0.0003 | N = 9 |
| Candida albicans | Fluconazole | 0.5 | 0.0 | N = 6 |
| ATCC 24433 | Nystatin | 4.8 | 1.6 | N = 9 |
| | Gentian Violet | 0.3 | 0.0 | N = 6 |
| | Oral rinse 150528 | .002 | .001 | N = 9 |
| Candida glabrata | Fluconazole | 7.3 | 1.6 | N = 6 |
| ATCC 15126 | Nystatin | 2.7 | 0.7 | N = 9 |
| | Gentian Violet | 1.02 | .32 | N = 6 |
| | Oral rinse 150528 | 0.0008 | 0.0002 | N = 9 |
| Issatchenkia | Fluconazole | 24 | 9 | N = 6 |
| orientalis | Nystatin | 5.4 | 1.3 | N = 9 |
| ATCC 6258 | Gentian Violet | 0.3 | 0.0 | N = 6 |
| (formerly known as | Oral rinse 150528 | 0.03 | 0.01 | N = 9 |
| Candida krusei) | | | | |
| Candida | Fluconazole | 1.2 | .4 | N = 6 |
| parapsilosis | Nystatin | 2.7 | 0.7 | N = 9 |
| ATCC 22019 | Gentian Violet | 0.2 | 0.0 | N = 6 |
| | Oral rinse 150528 | 0.03 | 0.03 | N = 9 |
| Candida | Fluconazole | 0.5 | 0.0 | N = 6 |
| tropicalis | Nystatin | 2.2 | 0.8 | N = 9 |
| ATCC 1369 | Gentian Violet | 0.3 | 0.2 | N = 6 |
| | Oral rinse 150528 | 0.14 | 0.03 | N = 9 |

Results show that the oral rinse has antifungal activity against five common Candida species. The oral rinse was three to several thousand times more potent than Fluconazole and fifteen to several thousand times more potent than Nystatin.

### Example 2

This experiment tested two examples of the oral rinse in the treatment of three different Candida organisms associated with oral mucositis. The embodiments of the Oral Rinses tested in this experiment were:
Oral Rinse 170915-10 μg/mL silver citrate complex, 4 mg/mL acemannan, 5 mg/mL Disodium EDTA, 0.1 mg/mL Hyaluronic acid, 2 mg/mL PVP, 5.75 mg/ml of masking and cherry flavor, purified water, pH adjustment with tris(hydroxymethyl)aminomethane to a pH of 6.36.
Oral Rinse 170719-8 μg/mL silver citrate complex, 3.9 mg/mL acemannan, 5 mg/mL Disodium EDTA, 0.1 mg/mL Hyaluronic acid, 2 mg/mL PVP, 5.75 mg/ml of masking agent and cherry flavor, purified water, pH adjustment with tris(hydroxymethyl)aminomethane to a pH of 6.38.

The antimicrobial activity of two oral rinse solutions as described above, Example 2, was tested using the microtube dilution method described in the Clinical and Laboratory Standards Institute (CLSI) Reference Method for Broth Dilution Antifungal Susceptibility Testing of Yeasts, M27-4. This method was used to determine the minimal inhibitory concentrations (MICs) for the antimicrobial agents listed in the Table below. In brief, a standard inoculum of each microorganism was prepared by diluting a 0.5 MacFarland turbidity standard of the microorganism 1:100 in RPMI media without sodium bicarbonate. Then, 100 μL of the standard inoculum was added to equal volumes of two-fold serial dilutions of the silver API (active pharmaceutical ingredient, silver citrate complex), controls, or the oral rinse solution, and the plates were incubated at 35° C. for 24 hours. MICs were determined photometrically as the lowest antimicrobial agent concentration (last well in the dilution series) that inhibited OD₅₃₀ absorbance at least 50%. Each experiment was performed three times, with each antimicrobial agent tested in duplicate or triplicate.

Table 2 below shows the average MIC for each of five tested products, two controls, two oral rinses and the API silver citrate complex alone, against each of three different Candida species.

TABLE 2

| | | | Standard | |
|---|---|---|---|---|
| MIC (mg/L) | | Mean | Deviation | |
| Candida albicans | Silver citrate complex | 0.234 | 0.000 | N = 6 |
| ATCC 24433 | Fluconazole | 0.583 | 0.714 | N = 6 |
| | Flucytosine | 0.250 | 0.137 | N = 6 |
| | Oral rinse 170915 | 0.001 | 0.001 | N = 9 |
| | Oral rinse 170719 | 0.002 | 0.001 | N = 9 |
| Candida | Silver citrate complex | 0.088 | 0.032 | N = 6 |
| parapsilosis | Fluconazole | 1.667 | 0.516 | N = 6 |
| ATCC 22019 | (Control Range- | | | |
| | 0.5-4.0) | | | |
| | Flucytosine | 0.063 | 0.000 | N = 6 |
| | (Control Range- | | | |
| | 0.06-0.25) | | | |
| | Oral rinse 170915 | 0.002 | 0.001 | N = 9 |
| | Oral rinse 170719 | 0.003 | 0.001 | N = 9 |
| Issatchenkia | Silver citrate complex | 0.117 | 0.000 | N = 6 |
| orientalis | Fluconazole | 14.667 | 3.266 | N = 6 |
| ATCC 5258 | (Control Range- | | | |
| (formerly known as | 8-64) | | | |
| Candida krusei) | Flucytosine | 3.667 | 0.816 | N = 6 |
| | (Control Range- | | | |
| | 4-16) | | | |
| | Oral rinse 170915 | 0.003 | 0.002 | N = 9 |
| | Oral rinse 170719 | 0.004 | 0.001 | N = 9 |

### Example 3

This experiment tested an example of the oral rinse in the treatment of Group A Strep that can be associated with oral mucositis. The embodiment of the Oral Rinse 141106 tested in this experiment included 10 μg/mL silver citrate complex, 4 mg/mL acemannan, 5 mg/mL Disodium EDTA, 0.1 mg/mL Hyaluronic acid, 2 mg/mL PVP, 5.75 mg/ml of masking and cherry flavor, purified water, pH adjustment with tris(hydroxymethyl)aminomethane to a pH of 6.38.

The antimicrobial activity of an oral rinse as described herein in Example 3, was tested using the microtube dilution method for determining minimal inhibitory concentrations (MIC) listed in the chart below. In brief, a standard inoculum of each microorganism was prepared by diluting a 0.5 MacFarland turbidity standard of the microorganism 1:100 in standard growth media. Then, 100 μL of the standard inoculum was added to equal volumes of two-fold serial dilutions of the oral rinse solution, and the plates were incubated at 35° C. for 48 hours. MICs were determined visually as the lowest antimicrobial agent concentration (last well in the dilution series) that inhibited growth. Each experiment was performed five times, with each organism tested in replicates of six. Mixtures demonstrating no growth remained clear while mixtures exhibiting growth turned cloudy.

Table 3 below shows the average MIC for oral rinse 141106 against Group A Strep and Candida spp.

TABLE 3

| | | |
|---|---|---|
| MIC (mg/L) Oral Rinse | Mean | SD |

| | | | |
|---|---|---|---|
| Streptococcus pyogenes | Oral rinse-141106 | 0.235 | .080 |
| (Grp A Strep) | N = 30 | | |
| Candida spp (Control) | | 0.282 | .064 |

### Example 4

This experiment tested an example of the oral rinse in the treatment of oral mucositis. The embodiment of the oral rinse 150528 tested in this experiment included 10 μg/mL silver citrate complex, 3 mg/mL acemannan, 5 mg/mL Disodium EDTA, 0.1 mg/mL Hyaluronic acid, 2 mg/mL PVP, 3.5 mg/ml of masking and cherry flavor, purified water, pH adjustment with tris(hydroxymethyl)aminomethane to a pH of 6.59.

The subject was a 69-year-old white female with diabetes and a 6-year history of reoccurring oral mucositis and candidiasis. She was eventually evaluated by an EENT and a biopsy was completed of her cheek and tongue. She was diagnosed with mucosal erosion, hyperparakeratosis, chronic mucositis and chronic candidiasis in 2011. Patient used dexamethasone rinse (0.5 mg/ml) two times per day for 6 years as needed for pain and inflammation and used chlorhexidine rinse as needed as well. She had also been treated for oral candidiasis with fluconazole twice. Patient could not eat anything spicy, crunchy, or carbonated during this time and could only use Biotene toothpaste. Her gums and tongue were swollen from blisters, and she was in considerable pain. Patient rated her pain on a scale from 1-10, with 10 being the worst, as an 8-10 at various times before using a rinse. Also, before using the oral rinse, the patient had blisters on the inside of her mouth, white streaks running from her gums to her lips, and her tongue was swollen and erythematous.

The patient's treatment regimen with the oral rinse was to swish and spit 10 milliliters three times a day for 7 days. However, after a couple days of treatment, patient reduced it to only twice a day to conserve what was left of the product. After treatment with the oral wash, patient experienced a 90% reduction in swelling and lesions. All of the patient's symptoms (blisters, swelling, and erythematous) resolved except the white streaks on her gums Immediately upon the first use of oral rinse, patient's pain reduced to a 5 where previously it was at an 8-10. Patient stated that she experienced more relief with the oral rinse than she had with any other previous treatment.

### Example 5

This experiment tested an example of the oral rinse in the treatment of recurrent oral ulceration. The embodiment of the oral rinse 141201 tested in this experiment included 5 μg/mL silver citrate complex, 3 mg/mL acemannan, 5 mg/mL Disodium EDTA, 0.1 mg/mL Hyaluronic acid, 2 mg/mL PVP, 3.5 mg/ml of masking and peppermint flavor, purified water, pH adjustment with tris(hydroxymethyl)aminomethane to a pH of 6.48.

The subject was a 52-year-old white female with a chronic history of reoccurring mouth ulcers. She was evaluated by her dentist and diagnosed with a dormant viral infection with outbreaks associated with stress and immune suppression. Patient used various treatments including steroids, antivirals and coating and protecting agents. Duration of ulcers usually persist for 7-10 days prior to clearing.

The patient's treatment regimen with the oral rinse was to swish and expectorate 10 milliliters three times a day for 2-3 days. The patient reported that ulcers cleared rapidly with reduction in inflammation and pain. On a pain scale of 0-10 with 10 being the most severe, the patient reported that pre-use pain was at a 5 and following use decreased to a 0-1 providing for excellent pain relief.

### Example 6

The purpose of this experiment was to determine the potency of one embodiment of the oral rinse on three common gram-positive cocci species tested: Staphylococcus aureus, Streptococcus pneumoniae, Streptococcus pyogenes by determining the minimal inhibitory concentrations (MIC) of the oral rinse on each species. The oral rinse was compared to vancomycin, a common antibiotic used to treat various infections.

The embodiment of the oral rinses (170915 and 171003) tested in this experiment included 10 ug/mL silver citrate complex, 4 mg/mL acemannan, 5 mg/mL Disodium EDTA, 0.1 mg/mL Hyaluronic acid, 2 mg/mL PVP, 3.5 mg/ml of masking and cherry flavor, purified water, and pH adjustment with tris(hydroxymethyl)aminomethane to a pH of 6.36 and pH of 6.40, respectively. The vancomycin control (Sigma-Aldrich, Cat: PHR1160-1G, Lot LRAA9717, 5120 ppm) was reconstituted 20.5 mg/4 mL RO water.

The antimicrobial activity of this embodiment of the oral rinse solution was tested using the microtube dilution method for determining minimal inhibitory concentrations (MIC) described in the Clinical and Laboratory Standards (CLSI) Methods for Dilution Antimicrobial Susceptibility Tests for Bacteria that Grow Aerobically, M07, 11th ed, Approved January 2018. In brief, a standard inoculum (125 μL of a 1:150 dilution of a 0.5 MacFarland turbidity standard) of each microorganism was prepared in the growth media recommended by CLSI M07 and added to equal volumes of two-fold serial dilutions of CelaCare OR, and the vancomycin control. MICs were determined visually and turbidometrically at OD530 nm as the highest dilution of CelaCare OR, or vancomycin (control) that showed at least half the turbidity, or 50% OD530 of the no drug control. Each experiment was performed three times, with each antimicrobial agent tested in triplicate. The MIC dilution was converted to μg/mL by multiplying the neat drug (Ag⁺) concentration by the dilution appropriate dilution factor. The results are shown in Table 4 below.

TABLE 4
MIC (mg/L) - CelaCare Oral Rinse and Vancomycin (Control)

| Organism | Device | Mean | SD |
|---|---|---|---|
| Staphylococcus aureus | N = 3 × 3 (9) | | |
| ATCC 29213 | OR - 170915 | 1.3 | 0.0 |
| Media - CAMHB | OR - 171003 | 1.2 | 0.0 |
| No LHB | Vancomycin (Control 0.5-2.0) | 0.7 | 0.2 |
| Streptococcus | N = 3 × 3 (9) | | |
| pneumoniae | OR - 170915 | 1.25 | 0.0 |
| ATCC 49619 | OR - 171003 | 0.81 | 0.48 |
| Media - CAMHB + LHB | Vancomycin (Control 0.06-0.5) | 0.13 | 0.04 |
| Streptococcus | N = 3 × 3 (9) | | |
| pyogenes | OR - 170915 | 0.2 | 0.1 |
| ATCC 12384 | OR - 171003 | 0.2 | 0.1 |
| Media - CAMHB + LHB | Vancomycin | 0.7 | 0.2 |

This example demonstrates that the oral rinses containing silver-citrate complex and acemannan (OR 170915 and 171003) demonstrate antibacterial activity against three common gram-positive cocci species tested: Staphylococcus aureus, Streptococcus pneumoniae, Streptococcus pyogenes. These oral rinses are 3.5× more effective than vancomycin (control) in its antimicrobial activity against Streptococcus pyogenes (the cause of strep throat).

### REFERENCES CITED

The following documents and publications are hereby incorporated by reference.

### RELATED U.S. PATENT DOCUMENTS

| | | |
|---|---|---|
| 4,735,935 | April 1988 | McAnalley |
| 4,851,224 | July 1989 | McAnalley |
| 4,966,892 | October 1990 | McAnalley |
| 4,959,214 | September 1990 | McAnalley |
| 4,957,907 | September 1990 | McAnalley |
| 4,917,890 | April 1990 | McAnalley |
| 5,118,673 | June 1992 | Carpenter |
| 5,106,616 | April 1992 | McAnalley |
| 5,308,838 | May 1994 | McAnalley |
| 5,441,943 | August 1995 | McAnalley |
| 5,487,899 | January 1996 | Davis |
| 5,703,060 | December 1997 | McAnalley et al. |
| 5,760,102 | June 1998 | Hall |
| 5,902,600 | May 1999 | Woller, et al. |
| 5,902,796 | May 1999 | Shand et al. |
| 6,274,548 | August 2001 | Ni et al. |
| 6,436,679 | August 2002 | Qiu et al. |
| 7,196,072 | March 2007 | Pasco et al. |
| 7,553,805 | June 2009 | Tichy et al. |
| 7,732,486 | June 2010 | Arata b |
| 7,842,317 | November 2010 | Kiani |
| 7,863,264 | January 2011 | Vange, et al. |
| 9,327,029 | May 2016 | Yates, et al. |

### FOREIGN PATENT DOCUMENTS

| | | |
|---|---|---|
| US 2008152697 | February, 2002 | US |
| WO 2006015317 | July 2004 | WO |
| GB 2028130 | March 1980 | GB |
| CA 1216520 | January 1987 | CA |
| RU 2317811 | February 2008 | RU |
| WO 03090799 | November 2003 | WO |
| EP 2704729 | April 2018 | EP |

### OTHER PUBLICATIONS

Aframian, D., Davidowitz, T., & Benoliel, R. (2006, July). The distribution of oral mucosal pH values in healthy saliva secretors. Oral Diseases, 12(4), 420-423. doi:10.1111/j.1601-0825.2005.01217.x
Ahmadi, A. (2012). Potential prevention: Aloe vera mouthwash may reduce radiation-induced oral mucositis in head and neck cancer patients. Chinese Journal of Integrative Medicine Chin. J. Integr. Med., 18(8), 635-640. doi:10.1007/s11655-012-1183-y
Bidra, A., BDS, M S, Tarrand, J., MD, Roberts, D., PhD, Rolston, K., MD, & Chambers, M., DMD, MS. (2011, April). Antimicrobial efficacy of oral topical agents on microorganisms associated with radiated head and neck Cancer patients: An in vitro study. Quintessence International, 42(4), 307-315.
Bhalang, K., Thunyakitpisal, P., & Rungsirisatean, N. (2013, May 7). Acemannan, a Polysaccharide Extracted from Aloe vera, Is Effective in the Treatment of Oral Aphthous Ulceration. The Journal of Alternative and Complementary Medicine, 19(5), 429-434. doi:10.1089/acm.2012.0164
Health and Consumer Protection Directorate, Scientific Committee on Consumer Products, Opinion on Citric acid and Silver citrate (2009, Jan. 21), General Report SCCP/1196/08.
Integrated Risk Information System. (2014, October). Retrieved Jun. 23, 2016, from http://www.epa.gov/iris/subst/0099.htm
Jettanacheawchankit, S., Sasithanasate, S., Sangvanich, P., Banlunara, W., & Thunyakitpisal, P. (2009, Jan. 27). Acemannan Stimulates Gingival Fibroblast Proliferation; Expressions of Keratinocyte Growth Factor-1, Vascular Endothelial Growth Factor, and Type I Collagen; and Wound Healing. J Pharmacol Sci Journal of Pharmacological Sciences, 109(4), 525-531. doi:10.1254/jphs.08204fp
Kong, E. F., Kucharíková, S., Dijck, P. V., Peters, B. M., Shirtliff, M. E., & Jabra-Rizk, M. A. (2015, Feb. 24). Clinical Implications of Oral Candidiasis: Host Tissue Damage and Disseminated Bacterial Disease. Infection and Immunity Infect. Immun., 83(2), 604-613. doi:10.1128/iai.02843-14
Lalla, R. V., Bowen, J., Barasch, A., Elting, L., Epstein, J., Keefe, D. M., . . . Elad, S. (2014). MASCC/ISOO clinical practice guidelines for the management of mucositis secondary to cancer therapy. Cancer, 120(10), 1453-1461. doi:10.1002/cncr.28592
Lansdown A B G. A pharmacological and toxicological profile of silver as an antimicrobial agent in medical devices. Adv Pharm Sci 2010; 2010:910686. Epub 2010 Aug. 24.
Lee, Y., PharmD., Atchley, D., PhD, Proctor, C., PharmD, Smith, F., PhD, Yi, S., PharmD, Loftis, C., MD candidate, & Yates, K., D V M. (2015, December). Time-kill Kinetics of a Novel Antimicrobial Silver Wound Gel Against Select Wound Pathogens. Wounds 2015, 27(12), 336-346.
Panghal, M., Kaushal, V., Kadayan, S., & Yadav, J. (2012, Jul. 20). Incidence and risk factors for infection in oral cancer patients undergoing different treatments protocols. BMC Oral Health, 12(1), 22. doi:10.1186/1472-6831-12-22
Poor, M. R., Hall, J. E., & Poor, A. S. (2002). Reduction in the incidence of alveolar osteitis in patients treated with the SaliCept Patch, containing Acemannan Hydrogel. Journal of Oral and Maxillofacial Surgery, 60(4), 374-379. doi:10.1053/joms.2002.31222
Roberts, D. B., & Travis, E. L. (1995). Acemannan-containing wound dressing gel reduces radiation-induced skin reactions in C3H mice. International Journal of Radiation Oncology*Biology*Physics, 32(4), 1047-1052. doi:10.1016/0360-3016(94)00467-y
Sonis, S. T., Eilers, J. P., Epstein, J. B., Leveque, F. G., Liggett, W. H., Mulagha, M. T., . . . Wittes, J. P. (1999, May 15). Validation of a new scoring system for the assessment of clinical trial research of oral mucositis induced by radiation or chemotherapy. Cancer, 85(10), 2103-2113. doi:10.1002/(sici)1097-0142(19990515)85:103.0.co;2-0 f
Stoopler, E., DMD, FDS RCSEd, FDS RCSEng, & Sollecito, T., DMD, FDS RCSEd. (2014). Oral Mucosal Diseases Evaluation and Management. Med Clin N Am, 98, 1323-1352.
Stuart, R., Lefkowitz, D., Lincoln, J., Howard, K., Gelderman, M., & Lefkowitz, S. (1997, Mar. 20). Upregulation of phagocytosis and candidicidal activity of macrophages exposed to the immunostimulant, acemannan. International Journal of Immunopharmacology, 19(2), 75-82. doi:10.1016/s0192-0561(97)00010-6
World Health Organization. (2004). Retrieved Jun. 3, 2016, who.int/en/

What is claimed is:

1. An oral rinse for the treatment of oral ulceration and oral mucositis comprising:
(a) silver citrate complex, wherein the silver citrate complex comprises a concentration of ionic silver of about 10 ppm;
(b) acemannan, wherein the acemannan has a concentration of about 0.4 w/w %;
(c) disodium EDTA;
(d) polyvinylpyrrolidone;
(e) hyaluronic acid;
(f) tris(hydroxymethyl)aminomethane, wherein the tris(hydroxymethyl)aminomethane has a concentration sufficient to produce a pH of about 6 to about 6.6 in the oral rinse.

2. The oral rinse of claim 1, further comprising flavoring agents.

3. The oral rinse of claim 1, further comprising purified water.

4. The oral rinse of claim 1, wherein the oral rinse comprises about 5 to about 10 μg/mL silver citrate complex and about 3 to about 4 mg/mL acemannan.

5. The oral rinse of claim 1, wherein the oral rinse comprises about 3 μg/mL silver citrate complex to about 15 μg/mL and about 1 mg/mL acemannan to about 10 mg/mL acemannan.

6. The oral rinse of claim 1, wherein the oral rinse comprises about 5 mg/mL disodium EDTA, about 2 mg/mL polyvinylpyrrolidone, and about 0.1 mg/mL hyaluronic acid.

7. A method for treating oral ulceration and oral mucositis in an oral cavity of a patient, comprising:
contacting the oral cavity of the patient with an amount of the oral rinse of claim 1 for a period of time.

8. The method of claim 7, wherein the amount of the oral rinse is about 10 mL.

9. The method of claim 7, wherein the period of time is about 30 seconds.

10. A method for reducing pain and time to heal in a patient affected by oral ulceration or oral mucositis, comprising:
contacting the oral cavity of the patient with an amount of the oral rinse of claim 1 for a period of time.

11. The method of claim 10, wherein the amount of the oral rinse is about 10 mL.

12. The method of claim 10, wherein the period of time is about 30 seconds.

13. A method for treating oral ulceration and oral mucositis in an oral cavity of a patient, comprising:
contacting the oral cavity of the patient with an amount of an oral rinse for a period of time, wherein the oral rinse comprises about 3 to about 15 μg/mL silver citrate complex, about 1 to about 10 mg/mL acemannan, disodium EDTA, polyvinylpyrrolidone, hyaluronic acid, and tris(hydroxymethyl)aminomethane.

14. The method of claim 13, wherein the oral rinse comprises about 5 mg/mL disodium EDTA, about 2 mg/mL polyvinylpyrrolidone, and about 0.1 mg/mL hyaluronic acid.

15. The method of claim 13, wherein the oral rinse comprises about 10 μg/mL silver citrate complex and about 4 mg/mL acemannan.

16. The method of claim 13, wherein the oral rinse further comprises a flavoring agents and purified water.

17. The method of claim 13, wherein the amount of the oral rinse is about 10 mL.

18. The method of claim 13, wherein the period of time is about 30 seconds.

19. A method for reducing pain and time to heal in a patient affected by oral ulceration or oral mucositis, comprising:
contacting the oral cavity of the patient with an amount of an oral rinse for a period of time, wherein the oral rinse comprises about 3 to about 15 μg/mL silver citrate complex, about 1 to about 10 mg/mL acemannan, disodium EDTA, polyvinylpyrrolidone, hyaluronic acid, and tris(hydroxymethyl)aminomethane.

20. The method of claim 19, wherein the oral rinse comprises about 5 mg/mL disodium EDTA, about 2 mg/mL polyvinylpyrrolidone, and about 0.1 mg/mL hyaluronic acid.

21. The method of claim 19, wherein the oral rinse comprises about 10 μg/mL silver citrate complex and about 4 mg/mL acemannan.

22. The method of claim 19, wherein the oral rinse further comprises a flavoring agents and purified water.

23. The method of claim 19, wherein the amount of the oral rinse is about 10 mL.

24. The method of claim 19, wherein the period of time is about 30 seconds.
